(19) United States
(12) Patent Application Publication
(10) Pub. No.: US 2025/0235238 A1
(43) Pub. Date: Jul. 24, 2025

(54) Angled Intraosseous Access System

(71) Applicant: Bard Access Systems, Inc., Salt Lake City, UT (US)

(72) Inventor: Daniel Pett, Sandy, UT (US)

(21) Appl. No.: 19/174,759

(22) Filed: Apr. 9, 2025

Related U.S. Application Data
(62) Division of application No. 17/410,863, filed on Aug. 24, 2021, now Pat. No. 12,274,469.
(60) Provisional application No. 63/069,988, filed on Aug. 25, 2020.

(51) Int. Cl. A61B 17/34; A61B 17/17; A61B 90/00

(52) U.S. Cl. CPC A61B 17/3403; A61B 17/17; A61B 17/3472; A61B 2017/3407; A61B 2017/3409; A61B 17/3494; A61B 2090/034

(57) ABSTRACT

An angled intraosseous access system includes a guide assembly including a guide block and a guide plate. The system can include a driver and a needle assembly rotatably coupled thereto. The guide assembly is configured to align the needle at a predetermined angle relative to the medullary cavity. Advantageously, the angled needle of the intraosseous access system can mitigate pain during infusion and mitigate backwalling.

### PRIORITY

This application is a division of U.S. patent application Ser. No. 17/410,863, filed Aug. 24, 2021, now U.S. Pat. No. 12,274,469, which claims the benefit of priority to U.S. Provisional Application No. 63/069,988, filed Aug. 25, 2020, each of which is incorporated by reference in its entirety into this application.

### BACKGROUND

Intraosseous (IO) access devices can provide vascular access by penetrating surface tissues and underlying hard bone cortex layers to place an access needle and provide access to a medullary cavity of a bone. Fluids and medications can flow through the needle lumen and into the medullary cavity to infuse into the vascular system. IO access events are typically performed in emergent situations where vascular access is rapidly required and/or direct venous access is not possible.

Patients who require IO access report experiencing increased pain during infusion rather than during penetration of the skin surface tissues or bone cortex. The cause of pain may be due to increased pressure in the medullary cavity due to the infused fluids and/or medication. Angling the needle during insertion may direct medication towards larger volumes of the medullary cavity, e.g. the tibial tuberosity, reducing the pressure within the medullary cavity, and in turn reducing discomfort. Further, angling the needle during penetration can mitigate “backwalling,” where the access needle traverses the medullary cavity and penetrates the far wall of bone cortex. Disclosed herein are angled intraosseous access systems including an angled guide plate and an angled guide block, and associated methods thereof that address the foregoing.

### SUMMARY

Disclosed herein is an angled intraosseous access system including, a needle assembly including a needle defining a needle axis and configured to access a medullary cavity of a bone, a driver rotatably coupled to the needle assembly and configured to advance the needle through a bone cortex to access the medullary cavity, and one or both of a guide plate and a guide block configured to engage a skin surface and align the needle axis at a predetermined angle relative to a longitudinal axis of the medullary cavity.

In some embodiments, the predetermined angle is between 1°-90° relative to the longitudinal axis of the medullary cavity. In some embodiments, one of the guide plate or the guide block includes a concave or convex skin engaging surface configured to engage the skin surface. In some embodiments, the guide plate is coupled to the guide block. In some embodiments, one of the guide plate or the guide block includes a guide recess configured to engage a fiduciary body part and align the needle with a target location. In some embodiments, the guide recess is disposed on a first portion that is slidably engaged with a second portion of one of the guide plate or the guide block which is coupled to the needle.

In some embodiments, the guide block includes a first series of notches and a second series of notches each configured to receive a portion of the driver, the first series of notches configured to align the needle axis with a first predetermined angle, the second series of notches configured to align the needle axis with a second predetermined angle different from the first predetermined angle. In some embodiments, the guide plate defines a channel configured to receive the needle therethrough and maintain the needle at the predetermined angle. In some embodiments, one of the guide plate or the guide block is releasably engaged with the driver body.

In some embodiments, one or both of the guide plate and the guide block includes an adhesive disposed on a surface thereof and configured to adhere to one or both of the driver and the skin surface. In some embodiments, a portion of the guide plate is configured to abut against a portion of the needle assembly to prevent a distal tip of the needle from backwalling a far wall of the medullary cavity.

Also disclosed is a method of accessing a medullary cavity including, engaging a skin engaging surface of a guide plate with a skin surface, aligning a needle of an intraosseous access system with a channel of the guide plate, the channel extending at a predetermined angle relative to the skin engaging surface, and advancing the needle through the channel to access the medullary cavity at the predetermined angle.

In some embodiments, the predetermined angle is between 1°-90° relative to a longitudinal axis of the medullary cavity. In some embodiments, the skin engaging surface includes a concave or a convex portion configured to engage the skin surface. In some embodiments, the method further includes coupling the guide plate with a guide block configured to engage the skin surface and a driver of the intraosseous access system and maintain an axis of the driver at the predetermined angle. In some embodiments, the method further includes engaging a guide recess, disposed on the skin engaging surface, with a fiduciary body part to align the needle with a target location.

In some embodiments, the method further includes sliding a first portion of the guide plate including the guide recess disposed thereon, relative to a second portion of the guide plate including the channel, to align the needle with a target location. In some embodiments, the method further includes adhering a portion of the guide plate to one of the driver or the skin surface. In some embodiments, the method further includes abutting a portion of a needle hub, coupled to the needle against a surface of the guide plate to prevent a distal tip of the needle from backwalling a far wall of the medullary cavity.

Also disclosed is a method of accessing a medullary cavity including, engaging a portion of a driver of an intraosseous access system with a surface of the guide block, engaging a skin engaging surface of the guide block with a skin surface, aligning an axis of a needle of the intraosseous access system with a predetermined angle, and advancing the needle along the needle axis to access the medullary cavity at the predetermined angle.

In some embodiments, the predetermined angle is between 1°-90° relative to a longitudinal axis of the medullary cavity. In some embodiments, the method further includes engaging a guide recess disposed on the skin engaging surface with a fiduciary body part to align the needle with a target location. In some embodiments, the method further includes sliding a first portion of the guide block including the guide recess disposed thereon, relative to a second portion of the guide block to align the needle with a target location. In some embodiments, the method further includes engaging the portion of the driver with one of a first series of notches or a second series of notches to align the axis of the needle axis with one of a first predetermined angle or a second predetermined angle, different from the first predetermined angle.

These and other features of the concepts provided herein will become more apparent to those of skill in the art in view of the accompanying drawings and following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates a perspective view of a guide plate angled IO access system, in accordance with some embodiments.

FIG. 1B illustrates close up detail of a needle engaged with an angled guide plate, in accordance with some embodiments.

FIG. 2 illustrates a side view of a guide block angled IO access system, in accordance with some embodiments.

FIG. 3 illustrates a side view of a combination angled IO access system including a guide plate and a guide block, in accordance with some embodiments.

FIG. 4 illustrates a side view of a combination angled IO access system including a guide block having a slidable portion, in accordance with some embodiments.

FIGS. 5A-5B illustrate side views of a guide block angled IO access system, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a needle disclosed herein includes a portion of the needle intended to be near a clinician when the needle is used on a patient. Likewise, a “proximal length” of, for example, the needle includes a length of the needle intended to be near the clinician when the needle is used on the patient. A “proximal end” of, for example, the needle includes an end of the needle intended to be near the clinician when the needle is used on the patient. The proximal portion, the proximal-end portion, or the proximal length of the needle can include the proximal end of the needle; however, the proximal portion, the proximal-end portion, or the proximal length of the needle need not include the proximal end of the needle. That is, unless context suggests otherwise, the proximal portion, the proximal-end portion, or the proximal length of the needle is not a terminal portion or terminal length of the needle.

With respect to “distal,” a “distal portion” or a “distal-end portion” of, for example, a needle disclosed herein includes a portion of the needle intended to be near or in a patient when the needle is used on the patient. Likewise, a “distal length” of, for example, the needle includes a length of the needle intended to be near or in the patient when the needle is used on the patient. A “distal end” of, for example, the needle includes an end of the needle intended to be near or in the patient when the needle is used on the patient. The distal portion, the distal-end portion, or the distal length of the needle can include the distal end of the needle; however, the distal portion, the distal-end portion, or the distal length of the needle need not include the distal end of the needle. That is, unless context suggests otherwise, the distal portion, the distal-end portion, or the distal length of the needle is not a terminal portion or terminal length of the needle.

Unless defined otherwise, all technical and scientific terms used herein have the same meaning as commonly understood by those of ordinary skill in the art.

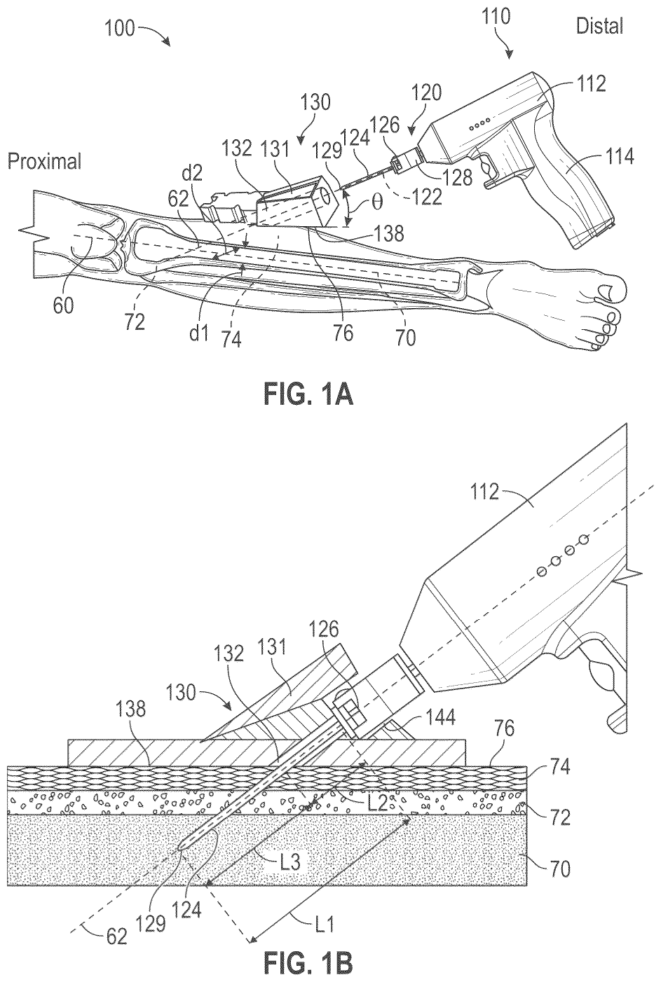

FIGS. 1A-1B show embodiments of a guide plate angled intraosseous (IO) access system 100 configured to penetrate a bone cortex 72 and surface tissues 74 to access a medullary cavity 70. In an embodiment, the guide plate angled IO access system 100 generally includes an IO access system 110 and an angled guide plate (“guide plate”) 130. The IO access system 110 generally includes a driver 112 having a body 114, and a needle assembly 120 rotatably coupled to the driver 112. In an embodiment, the driver 112 includes an automatic driver or a manual driver configured to rotate the needle assembly 120 and drill a needle 124 through the surface tissues 74 and bone cortex 72, access a medullary cavity 70 of a bone of a patient.

In an embodiment, the driver 112 can include a drive spring, electric motor, or similar electrical, mechanical, electro-mechanical, or kinetic mechanism configured to rotate the needle assembly 120 and drill the needle assembly into a bone of a patient. In an embodiment, the needle assembly 120 can include a needle 124 defining a lumen and supported by a needle hub 126. The needle assembly 120 can further include an obturator 122 disposed within the lumen of the needle 124 and supported by an obturator hub 128. The obturator 122 can be configured to prevent tissue and bone fragments from entering the needle lumen during a placement event and occluding a fluid flow through the needle lumen. Once the needle 124 has been placed, the driver 110 and obturator 122 can be removed. Fluids and/or medications can flow through the needle lumen and into the medullary cavity 70 to infuse into the vasculature of the patient. Further details and embodiments of IO access systems can be found, for example in the following published applications: US 2021/0093354, US 2021/0093355, US 2021/0093358, US 2021/0093356, US 2021/0093357, US 2021/0137558, WO 2018/075694, WO 2018/165334, and WO 2018/165339, each of which are incorporated by reference in its entirety herein.

In an embodiment, the guide plate 130 includes a body 131 defining a skin engaging surface 138, and a channel 132 extending at an angle (θ) relative to a longitudinal axis of the body 131. In an embodiment, the skin engaging surface 138 can be a bottom surface of the body 131. However, it will be appreciated that the skin engaging surface 138 can include one or more surfaces of the body 131. In an embodiment, the skin engaging surface 138 of the guide plate 130 can engage a skin surface 76 and align the channel 132 at an angle (θ) relative thereto. In an embodiment, the guide plate 130 align the channel 132 at an angle (θ) relative to a longitudinal axis 60 of the medullary cavity 70. In an embodiment, the angle (θ) of the channel 132 can be between 1° and 90°. In an embodiment, the angle (θ) of the channel 132 can be between 10°-75°. In an embodiment, the angle (θ) of the channel 132 can be substantially 45°, however greater or lesser angles are also contemplated.

In an embodiment, a diameter of the channel 132 can be equal to or slightly larger than a diameter of the needle 124. As such, the needle 124 can slidably engage the channel 132 and substantially align an axis 62 of the needle 124 with an axis of the channel 132. In some embodiments, the diameter of the guide plate channel 132 may be a consistent diameter along the length thereof. In some embodiments, the guide plate channel 132 or portion thereof may define a tapered shape, having a greater diameter proximate a first end relative to a second end, opposite the first end. In some embodiments, an entrance of the guide plate channel 132 may be tapered to facilitate aligning a needle 124 with the channel 132.

In an exemplary method of use, a user can engage a skin engaging surface 138 of the guide plate 130 with a skin surface 76 and align the channel 132 with a target insertion site. The user can then align the needle 124 with the channel 132 and advance a distal tip 129 of the needle 124 through the channel 132. Advancing the needle 124 through the channel 132 can align an axis 62 of the needle 124 with the axis of the channel 132. A user can then actuate the driver 112 to rotate the needle assembly 120 and “drill” the needle 124 into the patient. The needle 124 can then penetrate the surface tissues 74, hard bone cortex 72 and the medullary cavity 70 at the predetermined angle (θ) defined by the channel 132.

In an embodiment, the guide plate 130 can be coupled to the IO access system 110 and the guide plate 130 can be slidably engaged with the needle 124. As such, a user can support the skin engaging surface 138 against the skin surface 76 and the guide plate 130 can further support the IO access system 110 with the needle axis 62 aligned with the channel 132. The user can then actuate the driver 112 and urge the IO access system 110 including the needle 124 along the needle axis 62 at the predetermined angle (θ) defined by the channel 132. The needle 124 can slide through the channel 132 to penetrate the bone at the predetermined angle (θ), as described herein.

In an embodiment, the guide plate 130 can be coupled to the IO access system 110, and the needle assembly 120 can be slidably engaged relative to the IO access system 110 and guide plate 130 assembly. As such, a user can support the skin engaging surface 138 against the skin surface 76 and the guide plate 130 can further support the IO access system 110 with the needle axis 62 aligned with the channel 132. The user can then actuate the driver 112 and the IO access system 110 can urge the needle 124 along the needle axis 62 to penetrate the bone at the predetermined angle (θ), as described herein.

Advantageously, the insertion angle (θ) of the needle 124 can direct a fluid flow through the needle lumen towards the wider regions of the medullary cavity 70, for example at a head of the bone, and can reduce the pressure within the medullary cavity 70 as the fluids and/or medications are transfused. Advantageously, the guide plate 130 can be configured to align the needle 124 at an angle (θ) relative to the axis 60 of the medullary cavity 70 and can increase the penetrable depth (d) of the medullary cavity 70. As shown in FIG. 1A, for a given location within the medullary cavity 70, a perpendicular penetrable depth (d1) (i.e. when the needle is angled perpendicular to the axis 60 of the medullary cavity 70) is less than an angled penetrable depth (d2) (i.e. when the needle is angled (θ) relative to the medullary axis 60). As such, the angled penetrable depth (d2) mitigates the needle 124 from “backwalling” the far side of the medullary cavity 70. Further, in an embodiment, the guide plate 130 can be configured to align the needle 124 with the widest portion of the medullary cavity 70, e.g. at the head of the bone, as described in more detail herein.

In an embodiment, as shown in FIG. 1B, the length of the needle 124 extending between the needle hub 126 at a proximal end and a distal tip 129 can define a first length (L1). The channel 132 can define a second length (L2), less than the first length (L1), and extending axially. In an embodiment, the needle 124 can be advanced through the channel 132 until the distal tip 129 extends through the skin engaging surface 138 and a portion of the hub 126 abuts against a portion of the guide plate body 131, proximate the channel entrance 132. As such, with the skin engaging surface 138 contacting the skin surface 76, the distal tip 129 can extend through the channel 132 and penetrate the underlying tissues 74, bone cortex 72, etc. In an embodiment, the length (L2) of the channel 132 can be predetermined such that a portion of the needle 124 extends through the surface tissues 74, bone cortex 72 and into the medullary cavity 70 by a penetration length, or third length (L3). In an embodiment, the third length (L3) is the length of the needle (L1) less the length (L2) of the channel 132. Advantageously, the length (L1) of the needle 124 and the length (L2) of the channel 132 can be predetermined to ensure the penetration length (L3) of the needle 124 is less than a penetration length that would “backwall” the medullary cavity 70. This can prevent a user from over penetrating the needle 124 during an emergency placement event.

In an embodiment, as shown in FIG. 1B, the guide plate 130 can further include a needle hub slot 144 configured to receive a portion of the needle hub 144 therein. Advantageously, the needle hub slot 144 can be configured to facilitate aligning the axis 62 of the needle with axis of the channel 132. In an embodiment, the needle hub 126 can abut against a portion of the needle hub slot 144 to prevent the distal tip 129 of the needle 124 from extending beyond the predetermined penetrable length (L3), mitigating backwalling of the medullary cavity 70.

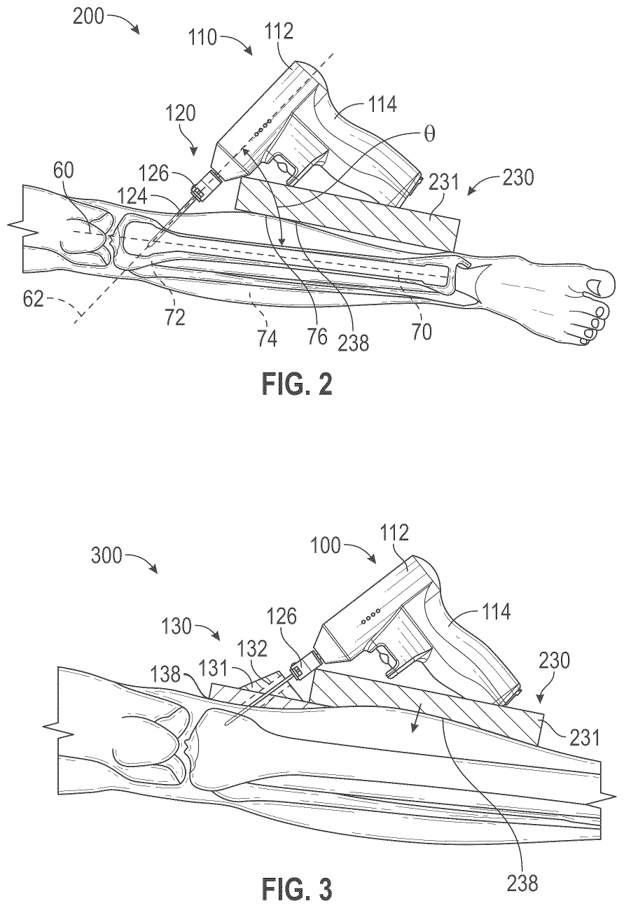

In an embodiment, as shown in FIG. 2, an angled IO access system 200 can include a guide block 230, configured to engage one or both of the IO access system 110 and the skin surface 76 and align the needle 124 at an angle (θ) relative to the skin surface 76 and/or an axis 60 of the medullary cavity 70. In an embodiment, the guide block 230 includes a body 231 having a skin engaging surface 238. In an embodiment, the skin engaging surface 238 can be a bottom surface of the body 231. However, it will be appreciated that the skin engaging surface 138 can include one or more surfaces of the body 231. Advantageously, the guide block 230 provides a stable surface for IO access system 110 to rest on.

In an embodiment, the guide block 230 can be integrally formed with the driver body 114. In an embodiment, the guide block 230 can be coupled to a portion of the driver body 114 using adhesive, bonding, welding, bolts, screws, or similar fasteners, or similar suitable means. In an embodiment, the guide block 230 can be releasably coupled with the driver body 114 using a clip, latch, interference fit, press-fit, or snap-fit engagement, or similar suitable mechanism.

In an embodiment, a user can grasp the driver body 114 with the guide block 230 coupled thereto, and engage the skin engaging surface 238 with the skin surface 76 of the patient. As such, an axis 62 of the needle 124 can be positioned at a predetermined angle (θ) relative to the axis 60 of the medullary cavity 70, as described herein.

In an embodiment, the IO access system 110 can be slidably engaged with the guide block 230 along the axis 62 of the needle 124. In an embodiment, the needle assembly 120 or portions thereof can be slidable relative to one or both of the driver 112 and the guide block 230, along the axis 62 of the needle 124. As such, the skin engaging surface 238 of the guide block 230 can engage the skin surface 76 and angle the axis 62 of the needle 124 relative to the axis 60 of the medullary cavity 70, as described herein. The needle 124 can then slide along the needle axis 62 through the surface tissues 74, bone cortex 72, and access the medullary cavity 70, as described herein.

In an embodiment, as shown in FIGS. 1A-2, one of the skin engaging surface 138 of the guide plate 130, or the skin engaging surface 238 of the guide block 230 can define a substantially flat surface. As shown in FIG. 3, in an embodiment, the skin engaging surface 138 of the guide plate 130, or the skin engaging surface 238 of the guide block 230 can include a regular or irregular concave or convex surface configured to substantially match a curved shape of a skin surface 76.

In an embodiment, as shown in FIG. 3, an angled IO access system 300 can include both the guide plate 130, and the guide block 230 to support and guide the IO access system 110 at a predetermined angle (θ) relative to the skin surface 76. In some embodiments, the guide plate 130 can be coupled to the guide block 230 to provide the user with a stable surface to rest a portion of the body 114 of the driver 112 and provide a guide for accessing the medullary cavity 70 at a predetermined angle (θ), as described herein.

In an embodiment, the guide plate 130 can be integrally formed with the guide block 230. In an embodiment, the guide plate 130 can be coupled to a portion of the guide block 230 using adhesive, bonding, welding, bolts, screws, or similar fasteners, or similar suitable means. In an embodiment, the guide plate 130 can be releasably coupled with the guide block 230 using a clip, latch, interference fit engagement, press-fit engagement, snap-fit engagement, hook, tab, slot, or similar suitable mechanism, or combinations thereof. Advantageously, the guide plate 130 and the guide block 230 being coupled together can offer increased stability during a medullary access event to ensure accurate placement of the access needle 124.

In an embodiment, one or more surfaces of the guide plate body 131 or the guide block body 231 can include an adhesive or the like. For example, the adhesive surface can be disposed on a skin engaging surface 138, 238 to facilitate securing the guide plate 130 or guide block 230 to the skin surface 76. In an embodiment, the adhesive surface can be configured to secure one or both of the guide plate 130 and the guide block 230 relative to the IO access system 110. In an embodiment, one or more surfaces of the guide plate 131 or the guide block body 231 can include a material, e.g. silicone rubber, or the like, having a high frictional coefficient to facilitate securing the guide plate 130 or guide block 230 relative to the skin surface 76, or to the IO access system 110, or combinations thereof.

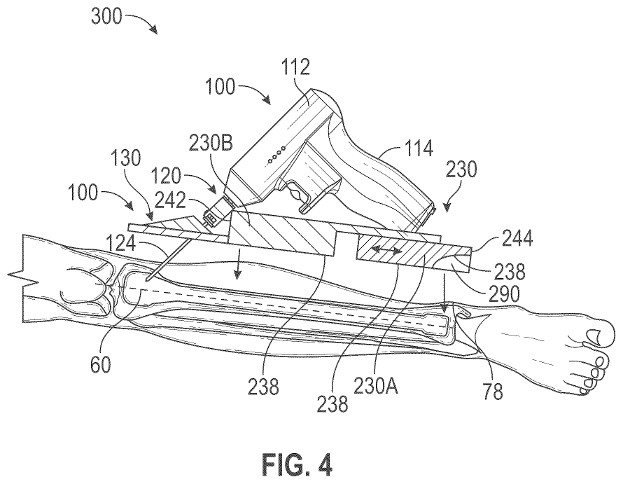

As shown in FIG. 4, in an embodiment, one of the guide plate 130 or the guide block 230 can include a guide recess 290 configured to engage a fiduciary body part 78 and to facilitate alignment of the needle 124 with a target area of bone. For example, a skin engaging surface 238 of the guide block 230 can include a guide recess 290 configured to receive the fiduciary body part 78, e.g. ankle bone, wrist bone, or the like, and to facilitate alignment of the needle 124 with a target area of bone. The driver 112 can be coupled to the guide block 230 with a needle assembly 120 disposed proximate a first end 242. The guide recess 290 can be disposed on a skin engaging surface 238 proximate a second end 244, disposed opposite the first end 242 along an axis of the guide block 230 extending substantially parallel to the skin surface 76 or the axis 60 of the medullary cavity 70. In an embodiment, the guide recess 290 can be a regular or irregular concave shape and can be configured to engage a fiduciary body part 78, e.g. an ankle bone or the like. When the guide block 230 is aligned with the fiduciary body part 78, the needle 124 of the IO access system 110 will be aligned at an angle (θ) with a target area of bone, e.g. a tibial tuberosity, or the like.

Advantageously, the guide block 230 including the guide recess 290 can quickly and intuitively align the needle 124 with a target area of the bone, e.g. the widest portion of the medullary cavity 70, during an emergency placement event where time and availability of trained personnel may be limited. Further, the guide block 230 can angle the needle 124 relative to the axis 60 of the medullary cavity 70 to further increase a penetrable depth for the needle 124.

In an embodiment, a portion of the guide plate 130 or the guide block 230 can be slidably engaged therewith, to allow a user to adjust the position of the guide recess 290 relative to the needle 124. For example, as shown in FIG. 4, a first portion 230A of the guide block 230 that includes the guide recess 290 can be slidably engaged with a second portion 230B of the guide block 230 that supports the IO access system 110. As such, the distance between the guide recess 290 and the needle 124 of the IO access system 110 can be modified to suit different size bones of different patients. For example, taller patients may have a greater distance between the ankle body and the tibial tuberosity than shorter patients.

In an exemplary method of use, the guide plate 130 and guide block 230 assembly can engage a skin surface 76 of a patient. In an embodiment, a concave or convex skin engaging surface 138, 238 can engage a curved skin surface 76 of the patient. In an embodiment, a guide recess 290 can engage a fiduciary body part 78 to align the needle 124 with a target area. In an embodiment, the user can adjust a first portion 230A of the guide block 230 relative to a second portion 230B and engage the guide recess 290 with the fiduciary body part 78. In an embodiment, the user can align the needle 124 with the channel 132. In an embodiment, the needle 124 can be “preloaded” within the channel 132 prior to the system 300 engaging the skin surface 76. The user can then actuate the driver 112 to rotate the needle assembly 120. In an embodiment, the IO access system 100 can slide relative to one of the guide plate 130 or the guide block 230 to advance the needle 124 through the channel 132. In an embodiment, the needle assembly 120 can advance through the channel 132 relative to the driver 112. The needle can then penetrate the bone at the predetermined angle (θ) to access the medullary cavity 70, as described herein.

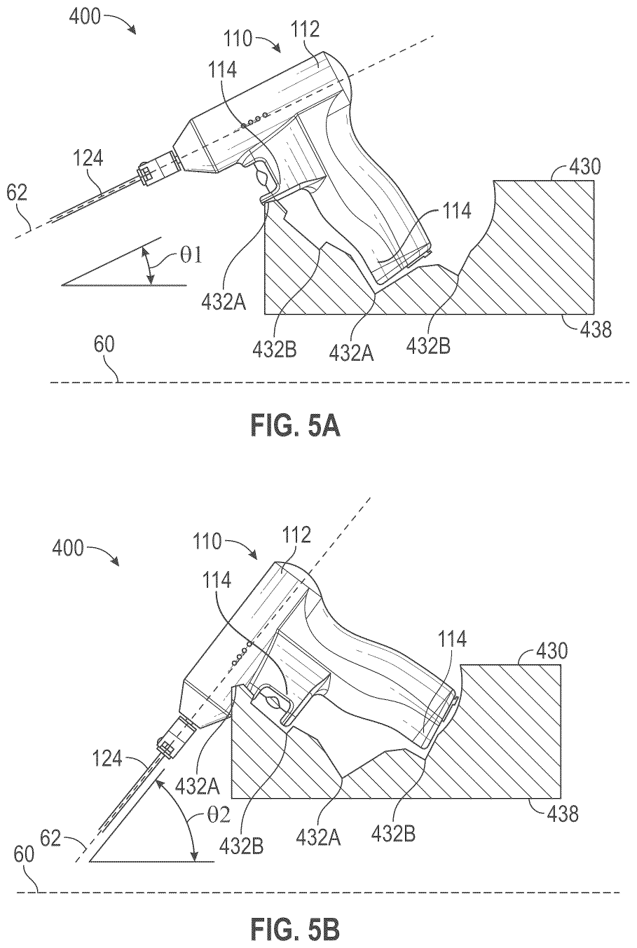

FIGS. 5A-5B show an embodiment of an adjustable angle IO access system 400 including a guide block 430 having a skin engaging surface 438 and one or more notches 432 disposed on one or more surfaces of the guide block 430. The one or more notches 432 can be configured to receive a portion of the driver body 114 and position the driver 112 at one or more predetermined angles relative to the axis 60 of the medullary cavity 70 when the skin engaging surface 438 engages a skin surface 76.

For example, as shown in FIG. 5A, a first series of notches 432A of the one or more notches 432 can receive one or more portions of the driver body 114 and position an axis 62 of the needle 124 at a first angle (θ1) relative to the skin surface 76 and/or medullary cavity axis 60. As shown in FIG. 5B, a second series of notches 432B of the one or more notches 432 can receive one or more portions of the driver body 114 and position an axis 62 of the needle 124 at a second angle (θ2) relative to the skin surface 76 and/or medullary cavity axis 60 different from the first angle (θ1). In an embodiment, one of the first angle (θ1) or the second angle (θ2) can be between 1°-90° relative to the axis 60 of the medullary cavity 70. In an embodiment, the guide block 430 can include one or more symbols, alphanumeric symbols, color codes, textures, or the like to indicate which series of notches 432 corresponds with which predetermined angles (θ1, θ2).

In an exemplary method of use, a skin engaging surface 438 of the guide block 430 can engage a skin surface 76 of the patient. The user can align a portion of the driver body 114 with one of the first series of notches 432A or the second series of notches 432B to selectively align an axis 62 of the needle 124 with one of the first angle (θ1) or the second angle (θ2). The user can then actuate the driver 112 to rotate the needle assembly 120 and advance the needle 124 into the medullary cavity 70, as described herein, at one of the first angle (θ1) or the second angle (θ2).

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An angled intraosseous access system, comprising:
a needle assembly including a needle configured to access a medullary cavity of a bone;
a driver rotatably coupled to the needle assembly and configured to advance the needle through a bone cortex to access the medullary cavity of the bone; and
a guide assembly comprising a guide plate coupled to a guide block, the guide assembly configured to align the needle at a predetermined angle relative to the medullary cavity.

2. The angled intraosseous access system according to claim 1, wherein the predetermined angle is between 1°-90° relative to a longitudinal axis of the medullary cavity.

3. The angled intraosseous access system according to claim 1, wherein the guide assembly includes a concave or convex surface configured to engage a skin surface over the medullary cavity.

4. The angled intraosseous access system according to claim 1, wherein the guide plate is integral with the guide block.

5. The angled intraosseous access system according to claim 1, wherein the guide block includes a first portion coupled to the guide block and a second portion slidable relative to the first portion to extend a length of the guide block.

6. The angled intraosseous access system according to claim 5, wherein the second portion of the guide block includes a guide recess configured to engage an anatomical landmark body part.

7. The angled intraosseous access system according to claim 1, wherein the guide block includes a first series of notches and a second series of notches each configured to receive a portion of the driver, the first series of notches configured to align the needle axis with a first predetermined angle, the second series of notches configured to align the needle axis with a second predetermined angle different from the first predetermined angle.

8. The angled intraosseous access system according to claim 1, wherein the guide plate defines a channel configured to receive the needle therethrough and maintain the needle at the predetermined angle.

9. The angled intraosseous access system according to claim 1, wherein the guide block is configured for releasable engagement with the driver.

10. The angled intraosseous access system according to claim 1, wherein the guide assembly includes an adhesive disposed on a surface of at least one of the guide plate and the guide block, the adhesive configured to adhere to one or both of the driver and a skin surface.

11. The angled intraosseous access system according to claim 1, wherein a portion of the guide plate is configured to abut against a portion of the needle assembly to prevent a distal tip of the needle from backwalling a far wall of the medullary cavity.

12. A method of accessing a medullary cavity, comprising:
positioning a guide assembly over the medullary cavity, the guide assembly comprising a guide plate coupled to a guide block;
aligning a needle of an intraosseous access system with a channel of the guide plate, the channel extending at a predetermined angle relative to the medullary cavity, wherein aligning the needle comprises sliding a first portion of the guide plate relative to a second portion of the guide plate; and
advancing the needle through the channel to access the medullary cavity at the predetermined angle.

13. The method according to claim 12, wherein the predetermined angle is between 1°-90° relative to a longitudinal axis of the medullary cavity.

14. The method according to claim 12, wherein the guide assembly includes a concave or a convex portion configured to engage the skin surface.

15. The method according to claim 12, wherein the second portion of the guide plate includes a guide recess, further comprising positioning the guide recess over an anatomical landmark body part.

16. The method according to claim 12, wherein the guide assembly comprises an adhesive on a surface, further comprising adhering a portion of the guide assembly to one or both of a driver of the intraosseous system and a skin surface.

17. The method according to claim 12, further comprising abutting a portion of a needle hub coupled to the needle against a surface of the guide plate to prevent a distal tip of the needle from backwalling a far wall of the medullary cavity.
